SEQUENCE LISTING (3 sequences)

SEQ ID NO: 1 - protein, 266 aa, Human
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 2 - protein, 267 aa, Human
MVKISFQPAVAGIKGDKADKASASAPAPASATEILLTPAREEQPPQHRSKRGGSVGGVCYLSMGMVVLLMGLVFASVYIYRYFFLAQLARDNFFRCGVLYEDSLSSQVRTQMELEEDVKIYLDENYERINVPVPQFGGGDPADIIHDFQRGLTAYHDISLDKCYVIELNTTIVLPPRNFWELLMNVKRGTYLPQTYIIQEEMVVTEHVSDKEALGSFIYHLCNGKDTYRLRRRATRRRINKRGAKNCNAIRHFENTFVVETLICGVV

SEQ ID NO: 3 - protein, 794 aa, Human
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

(19) United States
(12) Patent Application Publication
(10) Pub. No.: US 2010/0098682 A1
(43) Pub. Date: Apr. 22, 2010

(54) Effect of Bri Proteins on Ass Production

(76) Inventors: Luciano D'Adamio, Bronx, NY (US); Shuji Matsuda, Bronx, NY (US)

Correspondence Address: AMSTER, ROTHSTEIN & EBENSTEIN LLP, 90 PARK AVENUE, NEW YORK, NY, 10016, US

(21) Appl. No.: 11/921,976

(22) PCT Filed: Jun. 14, 2006
(86) PCT No.: PCT/US06/23135

§ 371 Date: Oct. 8, 2009

Related U.S. Application Data
(60) Provisional application No. 60/690,841, filed on Jun. 14, 2005.

(51) Int. Cl. A61K 38/48; C12N 5/071; A61K 38/00; G01N 33/53; A61K 31/7088; A61P 25/28

(52) U.S. Cl. 424/94.63; 435/325; 514/12; 435/7.1; 514/44 R

(57) ABSTRACT

Provided are methods for reducing inhibiting or preventing Aβ and/or AID production by a cell and methods of treating a subject having Alzheimer's disease. Also provided are methods of determining whether a compound is a mimic of a BRI2 or a BRI3. Additionally provided are pharmaceutical compositions of BRI2, BRI3 or furin, or vectors encoding those proteins.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grants AG22024-95264562 and AG21588-95264878, awarded by The National Institutes of Health.

### BACKGROUND OF THE INVENTION

(1) Field of the Invention

The present invention generally relates to control of Aβ production in Alzheimer's disease. More specifically, the invention is directed to the use of BRI proteins to inhibit γ-secretase cleavage of C99 and release of Aβ and/or APP intracellular domain (AID).

(2) Description of the Related Art

### REFERENCES CITED

Cao, X., and Sudhof, T. C. (2001) Science 293, 115-120.
Cupers, P., Orlans, I., Craessaerts, K., Annaert, W., and De Strooper, B. (2001) J Neurochem 78, 1168-1178.
Deleersnijder, W., Hong, G., Cortvrindt, R., Poirier, C., Tylzanowski, P., Pittois, K., Van Marck, E., and Merregaert, J. (1996) J Biol Chem 271, 19475-19482.
Gandy, S. (2005) J Clin Invest 115, 1121-1129.
Gianni, D., Zambrano, N., Bimonte, M., Minopoli, G., Mercken, L., Talamo, F., Scaloni, A., and Russo, T. (2003) J Biol Chem 278, 9290-9297.
Goate, A., Chartier-Harlin, M. C., Mullan, M., Brown, J., Crawford, F., Fidani, L., Giuffra, L., Haynes, A., Irving, N., James, L., and et al. (1991) Nature 349, 704-706.
Holton, J. L., Lashley, T., Ghiso, J., Braendgaard, H., Vidal, R., Guerin, C. J., Gibb, G., Hanger, D. P., Rostagno, A., Anderton, B. H., Strand, C., Ayling, H., Plant, G., Frangione, B., Bojsen-Moller, M., and Revesz, T. (2002) J Neuropathol Exp Neurol 61, 254-267.
Kang, J., Lemaire, H. G., Unterbeck, A., Salbaum, J. M., Masters, C. L., Grzeschik, K. H., Multhaup, G., Beyreuther, K., and Muller-Hill, B. (1987) Nature 325, 733-736.
Kieber-Emmons et al. (1997) Curr. Opin. Biotechnol. 8, 435-441.
Kim, S. H., Wang, R., Gordon, D. J., Bass, J., Steiner, D. F., Lynn, D. G., Thinakaran, G., Meredith, S. C., and Sisodia, S. S. (1999) Nat Neurosci 2, 984-988.
Kimberly, W. T., LaVoie, M. J., Ostaszewski, B. L., Ye, W., Wolfe, M. S., and Selkoe, D. J. (2003) Proc Natl Acad Sci USA 100, 6382-6387.
Kopan, R. (2002) J Cell Sci 115, 1095-1097.
Levy-Lahad, E., Wijsman, E. M., Nemens, E., Anderson, L., Goddard, K. A., Weber, J. L., Bird, T. D., and Schellenberg, G. D. (1995a) Science 269, 970-973.
Levy-Lahad, E., Wasco, W., Poorkaj, P., Romano, D. M., Oshima, J., Pettingell, W. H., Yu, C. E., Jondro, P. D., Schmidt, S. D., Wang, K., and et al. (1995b) Science 269, 973-977.
Maulik et al. (1997) Molecular Biotechnology: Therapeutic Applications and Strategies, Wiley-Liss, Inc.
Matsuda, S., Yasukawa, T., Homma, Y., Ito, Y., Niikura, T., Hiraki, T., Hirai, S., Ohno, S., Kita, Y., Kawasumi, M., Kouyama, K., Yamamoto, T., Kyriakis, J. M., and Nishimoto, I. (2001) J Neurosci 21, 6597-6607.
Passer, B., Pellegrini, L., Russo, C., Siegel, R. M., Lenardo, M. J., Schettini, G., Bachmann, M., Tabaton, M., and D'Adamio, L. (2000) J Alzheimers Dis 2, 289-301.
Pickford, F., Onstead, L., Camacho-Prihar, C., Hardy, J., and McGowan, E. (2003) Neurosci Lett 338, 95-98.
Pittois, K., Deleersnijder, W., and Merregaert, J. (1998) Gene 217, 141-149.
Ripka et al. (1998) Curr. Opin. Chem. Biol. 2, 441-452.
Rogaev, E. I., Sherrington, R., Rogaeva, E. A., Levesque, G., Ikeda, M., Liang, Y., Chi, H., Lin, C., Holman, K., Tsuda, T., and et al. (1995) Nature 376, 775-778.
Sanchez-Pulido, L., Devos, D., and Valencia, A. (2002) Trends Biochem Sci 27, 329-332.
Sanderson (1999) Med. Res. Rev. 19, 179-197.
Selkoe, D., and Kopan, R. (2003) Annu Rev Neurosci 26, 565-597.
Scheinfeld, M. H., Roncarati, R., Vito, P., Lopez, P. A., Abdallah, M., and D'Adamio, L. (2002) J Biol Chem 277, 3767-3775.
Scheinfeld, M. H., Ghersi, E., Davies, P., and D'Adamio, L. (2003) J Biol Chem 278, 42058-42063.
Sherrington, R., Rogaev, E. I., Liang, Y., Rogaeva, E. A., Levesque, G., Ikeda, M., Chi, H., Lin, C., Li, G., Holman, K., and et al. (1995) Nature 375, 754-760.
Sisodia, S. S., and St George-Hyslop, P. H. (2002) Nat Rev Neurosci 3, 281-290. Stagljar, I., Korostensky, C., Johnsson, N., and to Heesen, S. (1998) Proc Natl Acad Sci USA 95, 5187-5192.
Tanzi, R. E., Gusella, J. F., Watkins, P. C., Bruns, G. A., St George-Hyslop, P., Van Keuren, M. L., Patterson, D., Pagan, S., Kurnit, D. M., and Neve, R. L. (1987) Science 235, 880-884.
Thomas, G. (2002) Nature Rev. Mol. Cell. Biol. 3, 753-766. Vassar, R., Bennett, B. D., Babu-Khan, S., Kahn, S., Mendiaz, E. A., Denis, P., Teplow, D. B., Ross, S., Amarante, P., Loeloff, R., Luo, Y., Fisher, S., Fuller, J., Edenson, S., Lile, J., Jarosinski, M. A., Biere, A. L., Curran, E., Burgess, T., Louis, J. C., Collins, F., Treanor, J., Rogers, G., and Citron, M. (1999) Science 286, 735-741.
Vidal, R., Frangione, B., Rostagno, A., Mead, S., Revesz, T., Plant, G., and Ghiso, J. (1999) Nature 399, 776-781.
Vidal, R., Revesz, T., Rostagno, A., Kim, E., Holton, J. L., Bek, T., Bojsen-Moller, M., Braendgaard, H., Plant, G., Ghiso, J., and Frangione, B. (2000) Proc Natl Acad Sci USA 97, 4920-4925.
Vidal, R., Calero, M., Revesz, T., Plant, G., Ghiso, J., and Frangione, B. (2001) Gene 266, 95-102.
Zambrano, N., Buxbaum, J. D., Minopoli, G., Fiore, F., De Candia, P., De Renzis, S., Faraonio, R., Sabo, S., Cheetham, J., Sudol, M., and Russo, T. (1997) J Biol Chem 272, 6399-6405.

Amyloid precursor protein (APP) is a ubiquitous type I transmembrane protein (Kang et al., 1987; Tanzi et al., 1987) that undergoes a series of endoproteolytic events (Selkoe and Kopan, 2003; Sisodia and St. George-Hyslop, 2002). APP is first cleaved at the plasma membrane or in intracellular organelles by β-secretase (Vassar et al., 1999). While the ectodomain is released extracellularly (sAPPβ) or into the lumen of intracellular compartments, the COOH-terminal fragment of 99 amino acids (C99) remains membrane bound. In a second, intramembranous proteolytic event, C99 is cleaved, with somewhat lax site specificity, by the γ-secretase. Two peptides are released in a 1:1 stoichiometric ratio: the amyloidogenic Aβ peptide, consisting of 2 major species of 40 and 42 amino acids (Aβ40 and Aβ42, respectively) and an intracellular product named APP Intracellular Domain (AID or AICD) which is very short-lived and has been identified only recently (Passer et al., 2000; Cao and Sudhof, 2001; Cupers et al., 2001). In an alternative proteolytic pathway, APP is first processed by α-secretase in the Aβ sequence leading to the production of the solubleAPPα (sAPPα) ectodomain and the membrane bound COOH-terminal fragment of 83 amino acids (C83). C83 is also cleaved by the γ-secretase into the P3 and AID peptides. While Aβ is implicated in the pathogenesis of Alzheimer's disease, AID mediates most of the APP signaling functions. A pathogenic role for APP processing in AD has been ascertained by the finding that mutations in APP (Goate et al., 1991) and Presenilins (Sherrington et al., 1995; Levy-Lahad et al., 1995a,b; Rogaev et al., 1995), key components of the γ-secretase, cause autosomal dominant familial forms of AD. Thus, because of its biological and pathological importance, there is a need for understanding how APP cleavage is regulated. The present invention addresses that need.

### SUMMARY OF THE INVENTION

Accordingly, the inventors have discovered that BRI2 and BRI3 inhibits production of Aβ and APP intracellular domain (AID) from APP.

Thus, in some embodiments, the invention is directed to methods of reducing, inhibiting or preventing Aβ and/or AID production by a cell. The methods comprise contacting the cell with a BRI2 or BRI3 or a mimic thereof in an amount effective to reduce, inhibit or prevent Aβ and/or AID production by the cell.

In other embodiments, the invention is directed to additional methods of reducing, inhibiting or preventing Aβ and/or AID production by a cell. The methods comprise contacting the cell with a furin in an amount effective to reduce, inhibit or prevent Aβ and/or AID production in the cell.

Additionally, the invention is directed to methods of treating a subject having Alzheimer's disease. The methods comprise administering to the subject an amount of BRI2 or BRI3 or a mimic thereof effective to treat Alzheimer's disease in the subject.

In further embodiments, the invention is directed to other methods of treating a subject having Alzheimer's disease. The methods comprise administering to the subject an amount of a furin effective to treat Alzheimer's disease in the subject.

The invention is also directed to methods of determining whether a compound is a mimic of a BRI2 or a BRI3. The methods comprise combining the compound with a functional γ-secretase and a membrane-bound protein comprising a C99, then determining whether the compound inhibits cleavage of the C99 to release Aβ and/or AID. In these embodiments, the compound is a mimic of BRI2 or BRI3 if it inhibits the cleavage of the C99 by the γ-secretase.

In additional embodiments, the invention is directed to compositions comprising a purified BRI2 or BRI3 in a pharmaceutically acceptable excipient.

In further embodiments, the invention is directed to compositions comprising a purified furin in a pharmaceutically acceptable excipient.

The invention is also directed to compositions comprising a vector encoding a BRI2 or BRI3 in a pharmaceutically acceptable excipient.

In other embodiments, the invention is directed to compositions comprising a vector encoding a furin in a pharmaceutically acceptable excipient.

### BRIEF DESCRIPTION OF THE DRAWINGS

FIG. 1 is diagrams and photographs of western blots establishing that BRI2 is a ligand of APP. Panel a is a schematic representation of the BRI2 constructs used. The constructs are numbered 1 (amino acids 1 to 266, full length) and 2 (amino acids 1 to 131). Panels b-d shows western blots (WB) of anti-FLAG immunoprecipitates (IP αFLAG) and total lysates (TL) from HeLa cells expressing the indicated proteins show the specificity of BRI2/APP association and map the interaction sites. pc indicates the empty vector (pcDNA3.1); the numbers 1 and 2 indicate the BRI2 constructs shown in a; * indicates not-reduced anti-FLAG antibody; m. denotes mature, glycosylated forms of APP, while i. indicates the immature, unglycosylated APP. For western blots, αAPP represents the monoclonal antibody 22C11 while αAPPct is a rabbit polyclonal raised against the C-terminus of APP. Panel e is western blots of experiments where the lysates of HeLa cells transfected with both APP and BRI2 were precipitated with either a rabbit polyclonal control (RP) or αAPP-ct. Immunoprecipitates and total lysates were blotted with either the αAPP monoclonal antibody 22C11 or αFLAG. BRI2, as well as a ˜17 kDa BRI2 N-terminal fragment (BRI2nt) were precipitated by αAPPct together with APP.

FIG. 2 is photographs of western blots establishing that endogenous APP and BRI2 interact in the adult brain. In Panel a, lysates from HeLa cells transfected with FLAG-BRI2 were precipitated with a chicken control antibody (lane 3), a commercially available chicken αBRI2 antibody (lane 6), protein A/G beads alone (lane 9), a control rabbit polyclonal antibody (lane 12), the rabbit polyclonal EN3 αBRI2 antibody (lane 15), a distinct rabbit αBRI2 serum (lane 18) and a mouse αBRI2 polyclonal (lane 21). Total lysates (L), supernatants (S) and precipitants (P) were gel-separated and probed with αFLAG. Only EN3 was capable of precipitating BRI2. BRI2nt fragments of ˜17 and 14 kDa were not precipitated since the epitope recognized by EN3 is C-terminal to the Brichos domain. In Panel b, total brain homogenates were immunoprecipitated with either a control rabbit polyclonal (RP), αAPPct or EN3. Total brain lysate (T.L.) and immunoprecipitates were blotted with the αAPP monoclonal antibody 22C11 (top panel) or αAPPct (bottom panel).

FIG. 3 is graphs and photographs of western blots establishing that BRI2 regulates APP processing by secretases. Panels a-c, BRI2 reduces the APP-Gal4-driven luciferase activity in HeLa, N2a and HEK293 cells. Cells were co-transfected with APP-Gal4 together with pcDNA3.1 (pc) or FLAG-BRI2, BRI21-131 or BACE. Data are expressed as percentage of the luciferase activity measured in cells transfected with the empty vector. BRI2, BACE and pc transfected cells express similar levels of APP-Gal4 (not shown). Error bars represent ±SD for three independent experiments. Panel d, cells were transfected with either APP-Gal4, pcDNA3.1 (pc) or BRI2. Cells transfected with APP-Gal4 were than mixed at the indicated ratio with either BRI2 or pc.DNA3.1 transfected cells. Samples were analyzed for luciferase activity as described above 24 hours after transfection and mixing. Panels e-f, BRI2 inhibits production of Aβ40/Aβ42. HEK293 cells stably expressing APP (HEK293APP) were transfected with an empty vector (pc), BACE or FLAG-BRI2, and Aβ40 and Aβ42 secreted in the media were measured by ELISA. Aβ amount was normalized by the protein content of the lysates of the transfected cells. Error bars represent ±SD for three independent experiments. Panel g, pulse-chase experiment, representative of four independent experiments, of transfected HeLa cells. HEK293APP cells were transfected with an empty vector (vector) or FLAG-BRI2. The lysates of metabolically labeled cells were precipitated with αAPPct. The numbers above each lane indicates the hours the cells were chased (c). BRI2 expression decreases C83 production while dramatically increases the generation of C99. Panel h, The conditioned media of similarly transfected HEK293APP cells were harvested after 4 h labeling and were precipitated with either p21 or 6E10. While the amounts of sAPPα were decreased by BRI2, the total sAPP (sAPPα+sAPPβ) did not show a significant change indicating an augmentation of sAPPβ production and a shift of APP processing from α to β secretase. Panel i, Cells were transfected with APLP2 together with either pc.DNA3.1 or BRI2. 24 hours after transfection, cells were analyzed by western blot for BRI2 and APLP2 peptides.

FIG. 4 is photographs of western blots establishing that the first 102 amino acids of BRI2 are necessary to inhibit the efficient cleavage of C99 and that the same region is necessary for the binding of BRI2 to C99 of APP. The left panel shows western blots of total lysates (IT) from γ30 cells, which stably express APP and which were transfected with an empty vector (vec), myc-tagged full length BRI2 1-266 (BRI2), or various myc-tagged BRI2C-terminal deletions (indicated by the amino acids coded by the constructs). The right panel shows anti-myc immunoprecipitants (myc IP) of the corresponding lysates. HC indicates the heavy chain of myc antibody used in the immunoprecipitation.

FIG. 5 is a graph of APP-Gal4-driven luciferase activity establishing that the first 102 amino acids of BRI2 are all that is necessary to inhibit AID production. HEK293 cells were transfected with APP-Gal4 together with an empty vector or myc-tagged full length BRI2 or various BRI2 C-terminal deletion constructs (indicated by the amino acid ranges covered by the constructs). Data are expressed as percentage of the luciferase activity measured in cells transfected with the empty vector. Error bars represent ±SD for three independent experiments.

FIG. 6 is photographs of western blots establishing that BRI2 inhibits secretion of sAPPαsAPPβ. HEK293APP cells were transfected with an empty vector (−) or FLAG-BRI2 (+). sAPPα and sAPPβ were detected from the media of the transfected cells conditioned for 4 hours in Opti-MEM. APP and BRI2 expression was confirmed by the western blots of the total lysates of the transfected cells. APP expression does not change significantly.

### DETAILED DESCRIPTION OF THE INVENTION

The present invention is based in part on the inventors' discovery that BRI2 and BRI3 inhibits production of Aβ from APP. Since APP intracellular domain (AID) is concomitantly produced with Aβ, inhibiting production of Aβ also inhibits production of AID. Without being bound by any particular mechanism, it is believed that this inhibition of Aβ production is due to the inhibition of cleavage of APP by β-secretase and C99 by γ-secretase by BRI2 and BRI3, inhibiting the production of C99 and the release of Aβ from the C99. See Examples.

Thus, in some embodiments, the invention is directed to methods of reducing, inhibiting or preventing Aβ and/or AID production by a cell. The methods comprise contacting the cell with a BRI2 or BRI3 or a mimic thereof in an amount effective to reduce, inhibit or prevent Aβ and/or AID production by the cell.

In these embodiments, BRI2 and BRI3 are vertebrate integral membrane proteins that are also known as “integral membrane protein 2B” and “integral membrane protein 2C”, respectively. The human wild type forms of these proteins have the amino acid sequence of SEQ ID NO:1 and SEQ ID NO:2, respectively, with cDNA sequences provided as GenBank Accessions NM 021999 (BRI2) and NM 030926, NM 001012516, and NM 001012514 (three transcript variants of BRI3). Additionally, the BRI2 amino acid sequence for Macaque and the BRI3 amino acid sequence for mouse are known as GenBank Accessions Q60HCl and NP071862, respectively. With this and other known BRI2 and BRI3 information, the skilled artisan could determine the BRI2 and BRI3 sequence for any vertebrate using routine methods. Any vertebrate BRI2 and BRI3 protein would be expected to have an amino acid sequence at least 80% homologous to SEQ ID NO:1 and SEQ ID NO:2, respectively.

The inventors have also determined, by genetically synthesizing portions of the BRI2 protein, that a BRI2 protein consisting only of amino acids 1-102 is sufficient to reduce, inhibit or prevent Aβ and/or AID production. See Examples 1 and 2.

The BRI2 or BRI3 used in the present methods can also comprise peptidomimetics. As used herein, a peptidomimetic is a compound that is capable of mimicking a natural parent amino acid in a protein, in that the substitution of an amino acid with the peptidomimetic does not significantly affect the activity of the protein. Proteins comprising peptidomimetics are generally poor substrates of proteases and are likely to be active in vivo for a longer period of time as compared to the natural proteins. Many non-hydrolyzable peptide bond analogs are known in the art, along with procedures for synthesis of peptides containing such bonds. Non-hydrolyzable bonds include —CH₂NH, —COCH₂, —CH(CN)NH, —CH₂CH(OH), —CH₂O, CH₂S. In addition, peptidomimetic-containing peptides could be less antigenic and show an overall higher bioavailability. The skilled artisan would understand that design and synthesis of proteins comprising peptidomimetics would not require undue experimentation. See, e.g., Ripka et al. (1998); Kieber-Emmons et al. (1997); Sanderson (1999).

Thus, in preferred embodiments of these methods, the cell is contacted with a BRI2 or BRI3 that comprises amino acids and/or peptidomimetics equivalent to amino acids 1 to 102 of the human BRI2 protein having the sequence of SEQ ID NO:1 or the human BRI3 protein having the sequence of SEQ ID NO:2, wherein the BRI2 protein and the BRI3 protein has an amino acid sequence at least 80% homologous to SEQ ID NO:1 and SEQ ID NO:2, respectively.

In other preferred embodiments, the BRI2 or BRI3 is a naturally occurring protein. In some preferred embodiments, the BRI2 or BRI3 are more similar to the human BRI2 or BRI3 than 80% homologous. In those embodiments, the BRI2 or BRI3 preferably has an amino acid sequence at least 90% homologous to at least a portion of SEQ ID NO:1 or SEQ ID NO:2, respectively; more preferably at least 95% homologous to at least a portion of SEQ ID NO:1 or SEQ ID NO:2, respectively; and even more preferably, the BRI2 or BRI3 has an amino acid sequence at least 98% homologous to at least a portion of SEQ ID NO:1 or SEQ ID NO:2, respectively. In the most preferred embodiments, the BRI2 or BRI3 is 100% homologous to at least a portion of SEQ ID NO:1 or SEQ ID NO:2, respectively.

Since the BRI2 or BRI3 in these methods can consist of as few as the first 102 amino acids of those proteins, as used herein, “BRI2” or “BRI3” includes proteins that are smaller than the full length BRI2 or BRI3 proteins, e.g., as provided in SEQ ID NO:1 and SEQ ID NO:2. Thus, the proteins can be fewer than 250, 200, 150 or 125 amino acids and/or peptidomimetics. In other preferred embodiments, the BRI2 or BRI3 comprises amino acids and/or peptidomimetics equivalent to amino acids 1 to 102 of the human BRI2 or BRI3 protein having the sequence of SEQ ID NO:1 or SEQ ID NO: 2, respectively.

The BRI2 or BRI3 herein can also further comprise additional useful moieties, e.g., moieties that allow slow release or reduced degradation of the protein, such as scaffolding or PEG, or moieties that allow targeting to a particular cell type such as a nucleic acid sequence.

In these methods, the cell can be contacted with either a BRI2, a BRI3, or a mimic of a BRI2 or BRI3. As used herein, a mimic refers to any peptide or non-peptide compound that is able to mimic the biological action of a naturally occurring peptide, here BRI2 or BRI3, often because the mimic has a basic structure that mimics the basic structure of the naturally occurring peptide and/or has the salient biological properties of the naturally occurring peptide. Mimics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies or fragments thereof; non-proteinaceous portions of an isolated protein (e.g., carbohydrate structures); or synthetic or natural organic molecules, including nucleic acids and drugs identified through combinatorial chemistry, for example. Such mimics can be designed, selected and/or otherwise identified using a variety of methods known in the art. Various methods of drug design, useful to design mimics or other therapeutic compounds useful in the present invention are disclosed in Maulik et al., 1997, which is incorporated herein by reference in its entirety.

These methods are not limited to use with any particular cell, provided the cell is capable of producing Aβ and/or AID. Nonlimiting examples of cells that can be utilized with these methods are neurons and essentially any other mammalian cell that expresses APP either naturally or through genetic manipulation (see Example). The cell can also be neuronal-like or capable of differentiating into a neuron (e.g., a stem cell). In some preferred embodiments the cell is in a live mammal. Preferably, the mammal is an experimental model of Alzheimer's disease, or a human. In other preferred embodiments, the cell is a neuron in a live mammal, preferably a human. In the most preferred embodiments, the human has Alzheimer's disease or is at risk for acquiring Alzheimer's disease, such as someone that has a genetic predisposition to Alzheimer's disease.

The cell can be contacted with the BRI2 or BRI3 or mimic by any known method. Examples include directly applying the BRI2 or BRI3 or mimic, or administering the BRI2 or BRI3 or mimic to a mammal that is harboring the cell such that the BRI2 or BRI3 or mimic will travel to the cell, e.g., through the circulatory system or by crossing the blood-brain barrier. Where the BRI2 or BRI3 or mimic is a protein (i.e., a BRI2 or BRI3 protein), the cell can be contacted with a vector, such as a viral vector, comprising a nucleic acid sequence encoding at least a portion of a BRI2 or BRI3 protein, where the translation of the BRI2 or BRI3 encoded by the nucleic acid effects the contact. The latter method is a preferred method, particularly when the vector is capable of entering the cell (e.g., viral infection of the cell).

BRI2 and BRI3 are processed by furin and the product causes the inhibition of C99 processing. Therefore, an increase in furin in the cell also reduces, inhibits or prevents Aβ and/or AID production.

Thus, the present invention is also directed to additional methods of reducing, inhibiting or preventing Aβ and/or AID production by a cell. The methods comprise contacting the cell with a furin in an amount effective to reduce, inhibit or prevent Aβ and/or AID production in the cell.

Furin, or paired basic amino acid cleaving enzyme, is a cellular type-I transmembrane protein proprotein convertase (Thomas, 2002). The human wild type form of preprofurin has the amino acid sequence of SEQ ID NO:3, with a cDNA sequence provided as GenBank Accession NM002569. The mature protein has the sequence or amino acids 108-794 of SEQ ID NO:3. Additionally, the furin amino acid sequence for mouse is known as GenBank Accession NP035176. With this and other known information about vertebrate furins, the skilled artisan could determine the furin sequence for any vertebrate using routine methods. Any vertebrate furin protein would be expected to have an amino acid sequence at least 80% homologous to amino acids 108-794 of SEQ ID NO:3. In preferred embodiments, the furin comprises an amino acid sequence at least 95% identical to human furin having the sequence of amino acids 108-794 of SEQ ID NO:3; in the most preferred embodiments, the furin is a human furin.

The furin herein can also further comprise additional useful moieties, e.g., moieties that allow slow release or reduced degradation of the protein, such as scaffolding or PEG, or moieties that allow targeting to a particular cell type such as a nucleic acid sequence.

In these methods, the cell can be contacted with a compound that enhances the activity of native furin, for example a peptidase that converts profurin to furin, or a compound that enhances the transport of furin to sites where the BRI proteins are present. However; in preferred embodiments, the cell is contacted with furin protein, for example by administering the furin protein to a mammal that is harboring the cell such that the furin will travel to the cell, e.g., through the circulatory system or by crossing the blood-brain barrier. In more preferred embodiments, the furin is expressed from a vector that comprises a nucleic acid sequence encoding the furin protein. Preferably, these vectors are viral vectors that infect the cells, thus producing the furin protein in situ.

These methods are not limited to use with any particular cell, provided the cell is capable of producing Aβ and/or AID. Nonlimiting examples of cells that can be utilized with these methods are neurons and essentially any other mammalian cell that expresses APP either naturally or through genetic manipulation (see Example). The cell can also be neuronal-like or capable of differentiating into a neuron (e.g., a stem cell). In some preferred embodiments the cell is in a live mammal. Preferably, the mammal is an experimental model of Alzheimer's disease, or a human. In other preferred embodiments, the cell is a neuron in a live mammal, preferably a human. In the most preferred embodiments, the human has Alzheimer's disease or is at risk for acquiring Alzheimer's disease, such as someone that has a genetic predisposition to Alzheimer's disease.

In other embodiments, the invention is directed to methods of treating a subject having Alzheimer's disease. The methods comprise administering to the subject an amount of BRI2 or BRI3 or a mimic thereof effective to treat Alzheimer's disease in the subject.

In these methods, the subject is preferably administered a BRI2 or BRI3 that comprises amino acids and/or peptidomimetics equivalent to amino acids 1 to 102 of the human BRI2 protein sequence of SEQ ID NO:1 or the human BRI3 protein sequence of SEQ ID NO:2. In these embodiments, the BRI2 protein and the BRI3 protein has an amino acid sequence at least 80% homologous to SEQ ID NO:1 and SEQ ID NO:2, respectively. In other preferred embodiments, the BRI2 or BRI3 is a naturally occurring protein.

In these methods, the BRI2 or BRI3 or mimic thereof can be administered directly to the brain of the subject. Alternatively, the BRI2 or BRI3 or mimic thereof is administered in a manner that permits the BRI2 or BRI3 or a mimic thereof to cross the blood-brain barrier of the mammal. The BRI2 or BRI3 or mimic thereof can also be formulated in a pharmaceutical composition that enhances the ability of the BRI2 or BRI3 or mimic thereof to cross the blood-brain barrier of the subject.

Unless otherwise limited, pharmaceutical compositions in any embodiments described herein can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

In further embodiments, the invention is directed to other methods of treating a subject having Alzheimer's disease. The methods comprise administering to the subject an amount of a furin effective to treat Alzheimer's disease in the subject. The subject in these embodiments is preferably administered a furin that comprises amino acids and/or peptidomimetics equivalent to a human furin having the sequence of amino acids 108-794 of SEQ ID NO:3, where the furin has an amino acid sequence at least 80% homologous to SEQ ID NO:3. In other preferred embodiments, the furin is a naturally occurring protein, for example a human furin.

The furin in these embodiments can be administered directly to the brain of the subject. Alternatively, the furin can be administered in a manner that permits the compound to cross the blood-brain barrier of the mammal. The furin can also be formulated in a pharmaceutical composition that enhances the ability of the furin to cross the blood-brain barrier of the subject.

Using established methods for identifying mimics, the skilled artisan can identify mimics of BRI2 or BRI3 by identifying compounds the inhibit cleavage of a C99 to release Aβ and/or AID. Thus, the invention is also directed to methods of determining whether a compound is a mimic of a BRI2 or a BRI3. The methods comprise combining the compound with a functional 1-secretase and a membrane-bound protein comprising a C99, then determining whether the compound inhibits cleavage of the C99 to release Aβ and/or AID. In these embodiments, the compound is a mimic of BRI2 or BRI3 if it inhibits the cleavage of the C99 by the γ-secretase.

The inhibition of cleavage of the C99 to release Aβ and/or AID can be determined by any known methods, for example the methods described in the Example. Such methods include measuring release of Aβ and/or AID, e.g., using an Aβ and/or AID-specific antibody, where a BRI2 or BRI3 mimic would cause a reduction in Aβ and/or AID. Inhibition of C99 can also be determined by measuring changes in C99, where a mimic would cause an increase in C99 (see Example).

Also as established in the Example, inhibition of cleavage of C99 by BRI2 or BRI3 causes a decrease in the presence of C83 and sAPPα, and an increase in the presence of sAPPβ. Thus, a BRI2 or BRI3 mimic would causes a decrease in C83 and sAPPα and an increase in sAPPβ.

The above determinations can be made by any known method. Preferred methods include ELISA, mass spectroscopy or western blot. As is known in the art, western blotting allows more unequivocal identification of the compound than ELISA, but is a more time-consuming and cumbersome procedure.

These methods are not limited to any particular compounds to be evaluated. For example, a library of random compounds can be evaluated. Preferably, however, the compounds are designed to mimic a portion of the BRI2 or BRI3 protein comprising amino acids equivalent to amino acids 1 to 102 of the human BRI2 or BRI3 protein having the sequence of SEQ ID NO:1 or SEQ ID NO:2, respectively. Such compounds can be designed to mimic the three-dimensional structure and/or charge of the portion of the BRI2 or BRI3 protein, for example. Alternatively, the compound can comprise peptidomimetics such that the compound mimics the BRI2 or BRI3 amino acid sequence.

In preferred embodiments, the membrane-bound protein comprising a C99 is an amyloid precursor protein (APP), such as would occur in a cell expressing APP.

These methods can be performed in vitro (e.g., in a test tube). Preferably, however, the functional γ-secretase and membrane-bound protein comprising a C99 are in a live cell. Nonlimiting examples of such live cells are those that comprise a genetic construct that activates transcription of a reporter gene (e.g., luciferase) upon cleavage of a transgenic APP by γ-secretase, as in Example 1. In these embodiments, the transgenic APP preferably further comprises a Gal4 on the cytoplasmic domain of the transgenic APP (see Example).

Where the methods utilize a live cell, any cell that expresses a functional γ-secretase and an APP can be used. Non-limiting examples include neuronal cells or cells that produce a transgenic APP, such as an HEK293 cell, a HeLa cell, or an N2a cell. See Example.

In additional embodiments, the invention is directed to compositions comprising a purified BRI2 or BRI3 in a pharmaceutically acceptable excipient. Preferably, the BRI2 or BRI3 comprises amino acids and/or peptidomimetics equivalent to amino acids 1 to 102 of the human BRI2 protein having the sequence of SEQ ID NO:1 or the human BRI3 protein having the sequence of SEQ ID NO:2, where the BRI2 protein and the BRI3 protein has an amino acid sequence at least 80% homologous to SEQ ID NO:1 and SEQ ID NO:2, respectively. The BRI2 or BRI3 can comprise any number of amino acids and/or peptidomimetics from the full-length protein down to 102 amino acids and/or peptidomimetics, including, for example fewer than 250 amino acids and/or peptidomimetics, fewer than 200 amino acids and/or peptidomimetics, fewer than 150 amino acids and/or peptidomimetics, or fewer than 125 amino acids and/or peptidomimetics.

In some embodiments, the pharmaceutically acceptable excipient enhances the ability of the BRI2 or BRI3 to cross the blood-brain barrier of the subject. In other embodiments, the composition is formulated in unit dosage form for treatment of Alzheimer's disease.

The invention is additionally directed to compositions comprising a purified furin in a pharmaceutically acceptable excipient. Preferably, the furin comprises amino acids and/or peptidomimetics equivalent to a human furin having the sequence of amino acids 108-794 of SEQ ID NO:3, where the furin has an amino acid sequence at least 80% homologous to SEQ ID NO:3. Preferably, the furin is a naturally occurring protein.

In some embodiments, the pharmaceutically acceptable excipient enhances the ability of the furin to cross the blood-brain barrier of the subject. In other embodiments, the composition is formulated in unit dosage form for treatment of Alzheimer's disease.

The invention is also directed to compositions comprising a vector encoding a BRI2 or BRI3 in a pharmaceutically acceptable excipient. Preferably, the BRI2 or BRI3 comprises amino acids equivalent to amino acids 1 to 102 of the human BRI2 protein having the sequence of SEQ ID NO:1 or the human BRI3 protein having the sequence of SEQ ID NO:2, where the BRI2 protein and the BRI3 protein has an amino acid sequence at least 80% homologous to SEQ ID NO:1 and SEQ ID NO:2, respectively. In some embodiments, the pharmaceutically acceptable excipient enhances the ability of the BRI2 or BRI3 to cross the blood-brain barrier of the subject. In other embodiments, the composition is formulated in unit dosage form for treatment of Alzheimer's disease. These embodiments are not limited to any particular vector. However, in preferred embodiments, the vector is a virus.

The present invention is also directed to compositions comprising a vector encoding a furin in a pharmaceutically acceptable excipient. Preferably, the furin comprises amino acids equivalent to a human furin having the sequence of amino acids 108-794 of SEQ ID NO:3, where the furin has an amino acid sequence at least 80% homologous to SEQ ID NO:3. In other preferred embodiments, the furin is a naturally occurring protein. In some embodiments, the pharmaceutically acceptable excipient enhances the ability of the furin to cross the blood-brain bather of the subject. In other embodiments, the composition is formulated in unit dosage form for treatment of Alzheimer's disease. Preferably, the vector is a virus.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### The Protein Encoded by the Familial Dementia BRI2 Gene Binds APP and Inhibits Aβ Production

### Example Summary

Alzheimer's disease (AD), the most common senile dementia, is characterized by amyloid plaques, vascular amyloid, neurofibrillary tangles and progressive neurodegeneration. Amyloid is mainly composed by Aβ peptides, which derive from processing of the β-Amyloid Precursor Protein (APP) by secretases. The APP intracellular domain (AID), which is released together with AO, has signaling function since it modulates apoptosis and transcription. Despite its biological and pathological importance, the mechanisms regulating APP processing are poorly understood. Membrane-bound proteins prompt Notch cleavage by secretases and the release of a transcriptionally-active intracellular fragment. Considering the remarkable similitude between APP and Notch signaling, we have hypothesized that APP processing is similarly regulated. Here, we show that BRI2, a type II membrane protein, interacts with APP. Interestingly, 17 amino acids corresponding to the NH₂-terminal portion of Aβ are necessary for this interaction. Moreover, BRI2 expression regulates APP processing resulting in reduced Aβ and AID levels. Altogether, these findings characterize the BRI2-APP interaction as a regulatory mechanism of APP processing that inhibits Aβ production. Notably, BRI2 mutations cause Familial British (FBD) and Danish Dementia (FDD) that are clinically and pathologically similar to AD. Finding that BRI2 pathogenic mutations alter the regulatory function of BRI2 on APP processing would define dis-regulation of APP cleavage as a pathogenic mechanism common to AD, FDD and FBD.

### Introduction

As cleavage of other γ-secretase substrates is regulated by membrane bound ligands, we have postulated the existence of integral membrane proteins that bind APP and regulate its processing. Here, we describe BRI2 (Deleersnijder et al., 1996), a type II membrane protein that fulfills this description.

### Experimental Procedures

γ30 cells were maintained in DMEM supplemented with antibiotics and 10% fetal bovine serum as described (Kimberly et al., 2003).

Split-ubiquitin yeast two hybrid screening. The split-ubiquitin system provides an attractive alternative to analyze interactions between integral membrane proteins (Stagljar et al., 1998). The split-ubiquitin system and human brain libraries were purchased from Dualsystems Biotech (Zurich, Switzerland). The screenings were performed according to the manufacturers protocol. Briefly, human APP (amino acids 1-695), human APP (amino acids 1-664; APPNcas), or human APLP2 were cloned into pTMV4, pAMBV4, pAMBV4 bait vectors respectively, to obtain APP family bait proteins fused to the C-terminal half of ubiquitin (Cub), followed by a reporter fragment (LexA, a DNA-binding protein, fused to VP16, a transcriptional activation). Human brain libraries express proteins fused at the N-terminal half of mutated ubiquitin (NubG). For each library we screened approximately 5×10⁶ transformants. Clones coding for proteins that can interact with APP/APLP2-Cub, will promote the NubG:Cub interaction followed by recruitment of ubiquitin-specific protease(s), cleavage of the APP/APLP2-Cub bait, release of the LexA-VP16 transcription factor and the transcriptional activation of the two reporter genes (LacZ and HIS3). Library plasmids were recovered from HIS3 and LacZ positive yeast transformants and cloned into pcDNA3.1 with an N-terminal FLAG tag, and directly tested its ability to interact with APP by immunoprecipitation as described below. Screening for co-activator of both reporter genes resulted in the identification of known APP/APLP2-binding proteins, such as Fe65 (Zambrano et al., 1997).

Plasmids. Full-length BRI2 and BRI2¹⁻¹³¹ was PCR amplified from the two-hybrid clone and cloned into pcDNA3.1-FLAG (Matsuda et al., 2001). Mammalian expression vectors APP, APPNcas were described (Scheinfeld et al., 2002). A myc-tag was inserted after signaling sequence of ApoER2 and cloned into pEF-BOS. BACE was cloned from mouse brain cDNA and C-terminally myc tagged by cloning into pcDNA3mycHisB (Invitrogen).

Antibodies. The following antibodies are used: αFLAG (mouse monoclonal M2, Sigma); αAPP mouse monoclonals 22C11 (Chemicon) 6E10 (Signet labs) and p2-1 (Biosource); αmyc (mouse monoclonal 9E10, Santa Cruz Biotechnology); rabbit polyclonal antibody αAPPct (ZMD.316, Zymed) (Scheinfeld et al., 2002); chicken control antibody (IgY, Southern Biotechnology); chicken αBRI2 (IgY, BMA Biomedicals); Rabbit polyclonal control antibody (IgG, Southern Biotechnology); EN3 (Pickford et al., 2003) (rabbit polyclonal antibody); a rabbit αBRI2 (a gift from Dr. Jorge Ghiso), a mouse polyclonal was raised against a peptide encompassing the cytoplasmic tail of human BRI2. The rabbit polyclonal anti-APLP1 and anti-APLP2 C-terminal antibodies were purchased from Calbiochem.

Cell culture and transfection. HEK293, HEK293 stably expressing APP (HEK293APP), HeLa, N2a cells were maintained in Dulbecco's modified Eagle's medium. (DMEM) supplemented with penicillin, streptomycin, and 10% fetal bovine serum in 5% CO₂ at 37° C. FuGENE 6 (Roche Applied Science) or Metafectene (Biontex) was used for transfection.

Immunoprecipitation and western blot. Unless otherwise noted, all immunoprecipitation procedures were performed at 4° C. The transfected cells were lysed in Buffer A [20 mM Hepes/NaOH pH 7.4, 1 mM EDTA, 1 mM DTT, 150 mM NaCl, 0.5% (w/v) TritonX-100] containing 10% (v/v) Glycerol for 30 min, and the lysates were cleared at 20,000 g for 10 min. For FLAG immunoprecipitation, the cleared lysates were mixed with 20 μl of FLAG-M2 beads (Sigma) for 2 hours, and washed three times with Buffer A. The precipitates were boiled in 60 μl of 2×SDS sample buffer and subjected to western blot. For other immunoprecipitation, the cleared lysates were incubated with antibodies for one hour, and mixed with 20 μl of protein A/G beads (Pierce), washed and processed as above. Human brains (a generous gift of Dr. Peter Davies) were homogenized in Buffer A containing 10% (v/v) Glycerol using a Dounce homogenizer. The proteins were extracted overnight with the protein concentration at 5 mg/ml. Extracted proteins were cleared at 20,000 g for one hour. The supernatants were incubated with the indicated antibodies and protein A/G beads blocked with PBS containing 1% (w/v) BSA. Precipitants were washed and processed as described above.

Metabolic labeling. HEK293APP cells transfected with pcDNA3 or BRI2 were incubated in DMEM without methionine and cysteine (Invitrogen) supplemented with penicillin, streptomycin, and 10% fetal bovine serum, for 2 hours. Cells were then labeled 30 min by adding [³⁵S] labeled methionine and cysteine (ICN) to the culture media. The labeled cells were washed extensively, chased in DMEM supplemented with penicillin, streptomycin, and 10% fetal bovine serum for indicated periods of time. After the chase, cells were lysed and immunoprecipitated with αAPPct as described above. Labeled cells were cleared of medium by centrifugation at 20,000 g for 10 min, and were then immunoprecipitated with the indicated antibodies.

Luciferase assays. The assays were performed as described (Scheinfeld et al., 2003), except that APP-Gal4 fusion (Gianni et al., 2003) was used as a Gal4 source. Luciferase activity was normalized by the activity of β-galactosidase co-transfected to monitor the transfection efficiency.

Enzyme linked immunosorbent assay (ELISA). HEK293APP cells were transfected with pcDNA3 or BRI2. 24 hours after the transfection, the cells were conditioned for 24 hours, and Aβ40 and Aβ42 in the media were measured using human AβELISA kits (KMI Diagnostics), according to the manufacturer's protocol. The transfected cells were lysed and cleared as above and the amount of extracted protein was used to normalize the amount of Aβ detected by ELISA.

Protein determination. Protein concentrations were determined by Bio-Rad protein assay (Bio-Rad) and BSA as a standard.

### Results And Discussion

To test whether membrane-tethered proteins might regulate APP processing, we used the split-ubiquitin system to identify interactions between membrane proteins. Screening of a human brain cDNA library for proteins that interact with APP family proteins resulted in the identification of BRI2 (Deleersnijder et al., 1996) and BRI3 (Vidal et al., 2001) (not shown), members of a gene family of Type II membrane proteins containing a Brichos domain. Although the function of BRI proteins is unknown, BRI2 mutations are found in patients with FBD (Vidal et al., 1999) and FDD (Vidal et al., 2000). Of note, neuro-pathological findings in FBD and FDD include parenchyma pre-amyloid depositions (FBD and FDD) and plaques (FBD), neurofibrillary tangles, congophilic amyloid angiopathy (CAA) and neurodegeneration, similar to AD. Hence, because mutations in BRI2 cause AD-like familial dementia we further studied the physiological relevance of this BRI2-APP interaction.

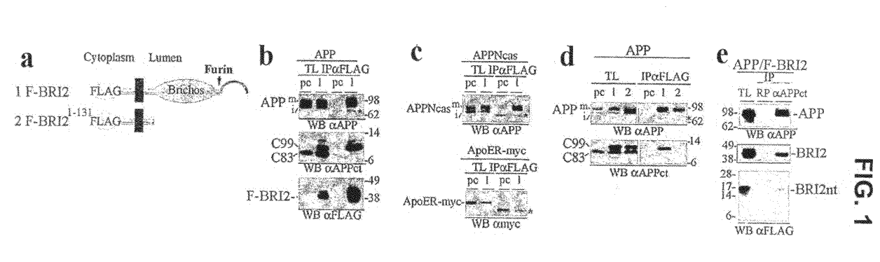

To assess the BRI2-APP interaction in mammalian cells, HeLa cells were co-transfected with BRI2 and APP constructs (FIG. 1a). Immunoprecipitation of cell lysates with an αFLAG antibody showed that BRI2 interacted with full length APP (FIGS. 1b and d), C99 (FIGS. 1b and d) and APPNcas, which present a deletion of most of the intracellular region of APP (FIG. 1c), but not C83 (FIGS. 1b and d). APP runs as a doublet. The lower APP band represents not glycosylated, immature APP; the upper form is instead composed of mature, glycosylated APP. Of note, only the mature, glycosylated forms of APP and APPNcas interacted with BRI2 (FIGS. 1b, c and d). It should also be noted that BRI2 overexpression dramatically increases the amounts of C99 (FIGS. 1b and 1d). The significance of this finding will be explored later.

Deletion of most of the BRI2 ecto-domain did not abolish the binding to APP (BRI2¹⁻¹³¹, FIG. 1d). The reverse immunoprecipitation with an αAPP antibody revealed that APP immuno-precipitates BRI2 (FIG. 1e). Additionally, a proteolytic ˜17 kDa BRI2NH₂-terminal fragment detected in transfected HeLa cells (BRI2nt, which is similar in size to BRI2¹⁻¹³¹) was also precipitated with APP (FIG. 1e). The specificity of these interactions was further supported by the evidence that BRI2 did not bind to ApoER2, another type I integral membrane protein (FIG. 1c). These findings attest that while the intracytoplasmic tail of APP and most of the APP and BRI2 ectodomain are dispensable for BRI2/APP interaction, a 17 amino acid region in the ectodomain of APP, juxtaposed to the transmembrane region and containing the NH₂-terminal Aβ sequence, is essential for this binding. These data strongly suggest that BRI2 and APP do not interact in trans (i.e. as receptor/ligand expressed on distinct membranes) but, rather, form a molecular complex in cellular membranes.

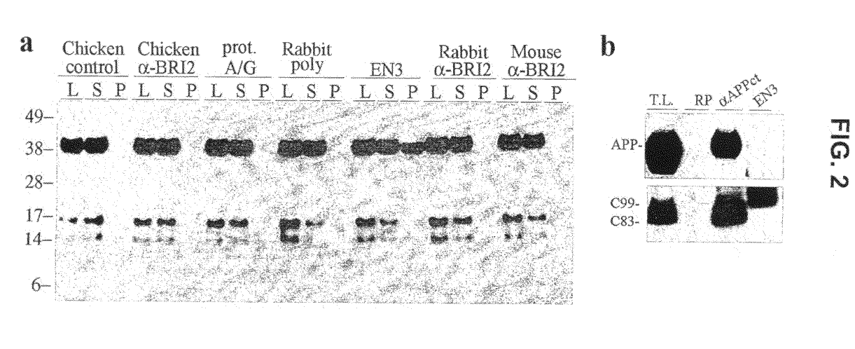

APP and BRI2 are both expressed in mature neural tissues. We therefore sought to determine if APP also interacts with BRI2 in the adult human brain. First, we tested four anti-BRI2 antibodies to determine whether they could immunoprecipitate human BRI2. For these tests, HeLa cells were transfected with FLAG-BRI2 and immunoprecipitated with the four BRI2 antibodies and controls. As shown in FIG. 2a, only the EN3 anti-BRI2 antibody was able to precipitate BRI2. Next, we made homogenates of human brains and performed immunoprecipitation with either the αAPPct antibody or EN3. As shown in FIG. 2b, C99 (and larger COOH-terminal APP fragments) was precipitated with both anti-APP as well as EN3 antibodies, while C99 was not precipitated with a rabbit polyclonal IgG. Of interest, also in this case C83 did not precipitate with BRI2, although it was precipitated by αAPPct. Moreover, full length APP was also precipitated by EN3, albeit at low levels. Altogether, these experiments indicate that endogenous APP and BRI2 associate in the adult human brain. In addition, they show that BRI2 preferentially interacts with C99 and larger APP C-terminal fragments but not with C83.

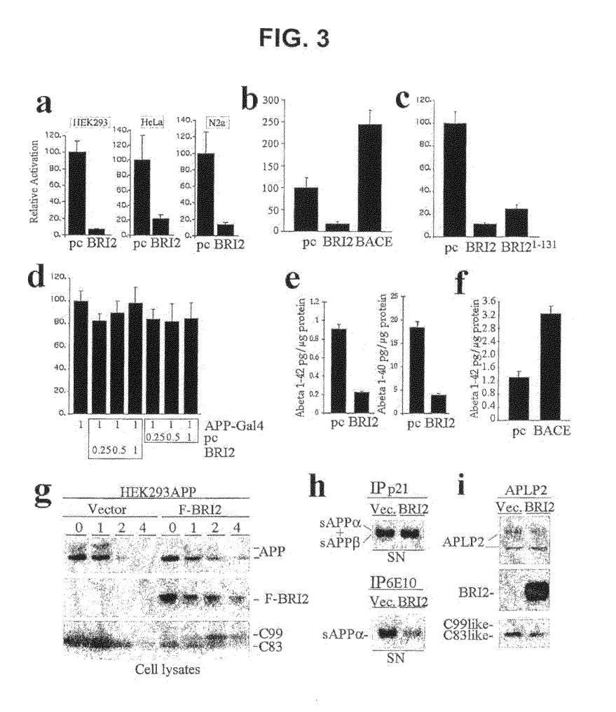

As shown in FIGS. 1b and 1d, expression of BRI2 constructs invariantly results in increased levels of C99. It is likely that this dramatic increase in C99 levels is dependent on an effect of BRI2 on APP processing. To directly test for this, we expressed FLAG-tagged BRI2 in HeLa, HEK293 and N2a cells together with APP-Gal4, a luciferase reporter under the control of a Gal4 promoter and a β-galactosidase construct. APP-Gal4 is a fusion of the yeast transcription factor Gal4 to the cytoplasmic domain of APP. γ-cleavage of APP-Gal4 releases AID-Gal4 from the membrane to the nucleus with consequent activation of luciferase transcription (Gianni et al., 2003). As shown in FIG. 3a, BRI2 reduces luciferase activity in all three cell lines, suggesting an inhibition of AID formation. Instead, transfection of β-secretase (BACE) resulted in increased AID release, as expected (FIG. 3b). The BRI2¹⁻¹³¹ mutant, that still interacts with APP and produces increased C99 levels (FIG. 1d), also inhibit AID release (FIG. 3c). Lastly, mixing experiments show that, for BRI2 to repress AID-Gal4 release, it must be co-expressed with APP-Gal4 in the same cell. In fact, mixing cells expressing BRI2 with cells expressing APP-Gal4 does not affect release of AID (FIG. 3d). This further suggests that BRI2 and APP interact in cis rather than in trans.

To further validate this system, we have measured Aβ in the conditioned media of HEK 293 transfected with BRI2 and found that BRI2 significantly diminished Aβ40 and Aβ42 levels (FIG. 3b). Again, BACE transfection increased Aβ40 and Aβ42 secretion (FIG. 3f).

Inhibition of AID and Aβ production by BRI2 suggests that BRI2 expression reduces cleavage of APP by the γ-secretase. However, it is also possible that BRI2 could modulate the β- and α-cleavage of APP. As discussed above, cleavage of APP by either α- or β-secretase releases sAPPβ and sAPPα in the supernatant, respectively. While increased amounts of either sAPPα or sAPPβ indicate increased α- or β-cleavage, reduction of either sAPPα or sAPPβ reflect decreased α- or β-cleavage. Thus, to determine whether BRI2 affects either α- or β-secretase, we measured the amounts of sAPPα and sAPPβ. In these same experiments, we also measured intracellular levels of C99 and C83. HEK293-APP cells were transfected with FLAG-BRI2 or a vector control. Transfected cells were pulse-labeled with [³⁵S]methionine-cysteine for 30 min, then chased for 0, 1, 2, and 4 hours at 37° C. (FIG. 3c). The cell lysates were immunoprecipitated with αAPP-ct antibody at each time point (FIG. 3c). To measure secreted APP (sAPPα and sAPPβ), supernatants were collected from cells labeled for 4 hours and precipitated with the anti-APP antibodies P21 (which precipitates both sAPPα and sAPPβ) or 6E10 (which only precipitates sAPPα) (FIG. 3d). BRI2 transfection resulted in decreased amounts of C83 (FIG. 3c) and sAPPα (FIG. 3d). Conversely, the levels of C99 (FIG. 3c) and sAPPβ (FIG. 3d) were augmented. Notably, BRI2 was co-immunoprecipitated by the αAPP-ct antibody at all time points. Thus, BRI2 expression reduces cleavage of APP by α-secretase while increases its processing by β-secretase. The concomitant inhibition of γ-secretase and increase in β-cleavage of APP explains the dramatic increase in C99 levels.

APP is a member of a family of proteins which include APLP1 and APLP2. APLP1 and APLP2 are also g-secretase substrates (Scheinfeld et al., 2002) and, among the numerous γ-secretase substrates are those that bear more sequence similarity to APP. Thus, to test whether BRI2 generally affects g-secretase or specifically inhibits g-cleavage of APP, we transfected BRI2 with either APLP1 or APLP2. Western blot using anti-APLP1 or anti-APLP2 C-terminal antibodies indicates that BRI2 expression does not promote accumulation of C-terminal fragments of APLP1 (not shown) and APLP2 (FIG. 3i). This result supports the notion that BRI2 specifically blocks the γ-activity on APP but not on other γ-substrates.

Altogether, these studies suggest that BRI2 and APP form a multimolecular complex in cell membranes. While the stoichiometry of APP and BRI2 in such complexes has to be investigated and whether BRI2 and APP are found in a structure comprising other proteins is unknown, our data suggest that BRI2 functions as an endogenous regulator of APP processing. More specifically, we found here that BRI2 expression decreases both α- and γ-cleavage of APP. Although the detailed molecular mechanisms responsible for these functions must be directly addressed, the finding that BRI2 interacts with a region of APP comprising the α- and γ-cleavage sites insinuates that BRI2 physically masks the two target sequences from the secretases.

Recently, mutations in BRI2 have been found in FBD (Vidal et al., 1999) and FDD (Vidal et al., 2000) patients. Both wild type and mutant BRI2 are processed by furin (Kim et al., 1999), this processing resulting in the secretion of a C-terminal peptide. Furin cleavage of wild type BRI2 releases a 17 aa-long peptide. In FBD patients, a point mutation at the stop codon of BRI2 results in a read-trough of the 3′-untranslated region and the synthesis of a BRI2 molecule containing 11 extra amino acids at the C-terminus. Furin cleavage of this mutated BRI2 generates a longer peptide, the ABri peptide, which is deposited as amyloid fibrils. In the Danish kindred, the presence of a 10-nt duplication one codon before the normal stop codon produces a frame-shift in the BRI2 sequence generating a larger-than-normal precursor protein, of which the amyloid subunit comprises the last 34 C-terminal amino acids. The deposition of ABri and ADan amyloid is considered the pathogenic cause of these dementia. However, the finding that BRI2 regulates APP processing is intriguing and prompts speculation that altered APP processing is also a pathogenic factor in FBD and FDD. Consistent with this hypothesis, in FDD patients elevated levels of Aβ42 deposition are detected together with ADan in CAA lesions.

### Example 2

### Further Studies on the Effect of BRI2 on App Processing

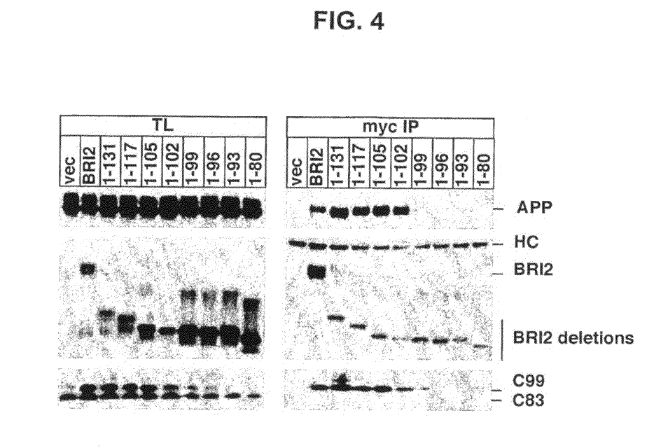

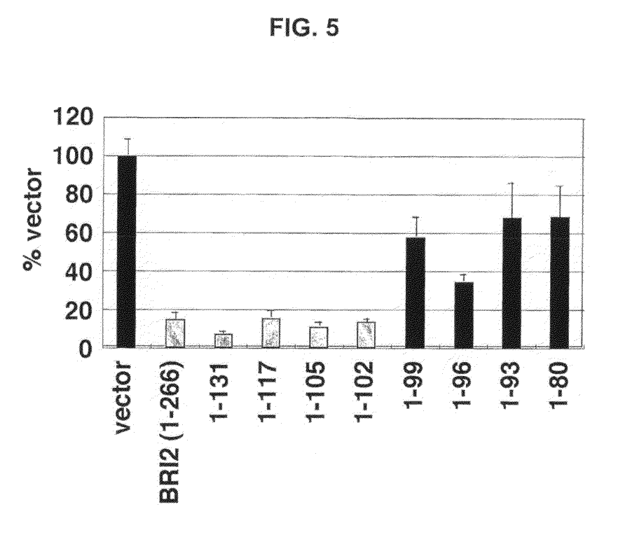

Using the methods described in Example 1, the effect of BRI2 deletion constructs consisting of the first 80, 93, 96, 99, 102, 105, 117, and 131 amino acids were measured. As shown in FIG. 4 (left panel), only the truncation larger than 99 amino acids showed the accumulation of C99 in the total lysates, which indicates the inhibition of further processing of C99. The deletion constructs consisting of the first 96 and 99 amino acids showed much poorer inhibitory effect, and those consisting of 80 and 93 did not display the inhibition. This inhibitory effect of BRI2 on C99 processing paralleled the binding of these BRI2 truncations to C99 and full length APP as shown FIG. 4 (the right panel). The same set of deletion constructs were used in determining their effect on AID production. The results of APP-Gal4-driven luciferase activity (FIG. 5) further support the conclusion that BRI2 constructs comprising more than the first 99 amino acids are required for the efficient inhibition of AID production.

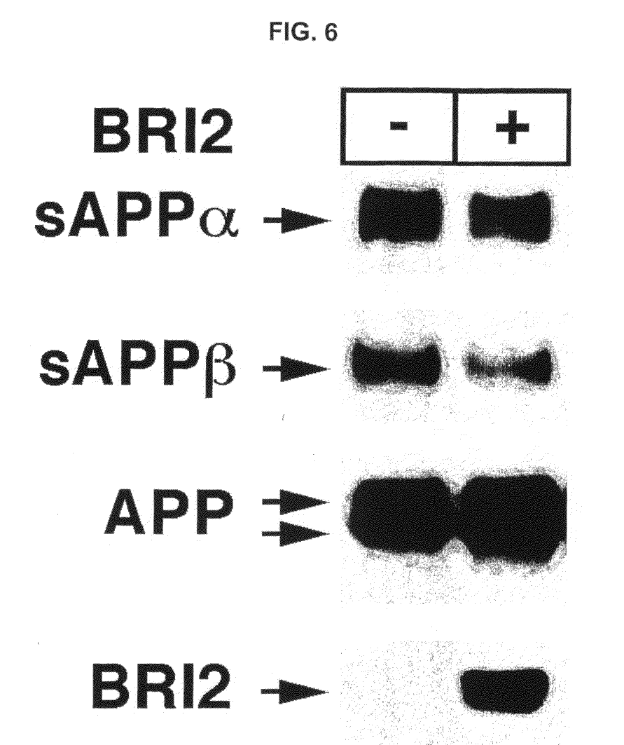

Example 1 established that sAPPα is reduced in the presence of BRI2, indicating that BRI2 inhibits α-secretase. Additional experiments were conducted to determine the effect of BRI2 on sAPPβ production. As shown in FIG. 6, sAPPβ production is also reduced in the presence of BRI2, indicating that BRI2 inhibits β-secretase.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

### SEQ ID NOs

### SEQ ID NO:1—Human BRI2 Amino Acid Sequence GenBank Q9Y287

| | |
|---|---|
| 1 | mvkvtfnsal aqkeakkdep ksgeealiip pdavavdckd pddvvpvgqr rawcwcmcfg |
| 61 | lafmlagvil ggaylykyfa lqpddvyycg ikyikddvil nepsadapaa lyqtieenik |
| 121 | ifeeeevefi svpvpefads dpanivhdfn kkltayldln ldkcyvipln tsivmpprnl |
| 181 | lellinikag tylpqsylih ehmvitdrie nidhlgffiy rlchdketyk lqrretikgi |
| 241 | qkreasncfa irhfenkfav etlics |

### SEQ ID NO:2—Human BRI3 Amino Acid Sequence GenBank Q9NQX7.

| | |
|---|---|
| 1 | mvkisfqpav agikgdkadk asasapapas ateilltpar eeqppqhrsk rggsvggvcy |
| 61 | lsmgmvvllm glvfasvyiy ryfflaqlar dnffrcgvly edslssqvrt qmeleedvki |
| 121 | yldenyerin vpvpqfgggd padiihdfqr gltayhdisl dkcyvielnt tivlpprnfw |
| 181 | ellmnvkrgt ylpqtyiiqe emvvtehvsd kealgsfiyh lcngkdtyrl rrratrrrin |
| 241 | krgakncnai rhfentfvve tlicgvv |

### SEQ ID NO:3—Human Furin Preproprotein GenBank NP002560

| | |
|---|---|
| 1 | melrpwllwv vaatgtlvll aadaqgqkvf tntwavripg gpavansvar khgflnlgqi |
| 61 | fgdyyhfwhr gvtkrslsph rprhsrlqre pqvqwleqqv akrrtkrdvy qeptdpkfpq |
| 121 | qwylsgvtqr dlnvkaawaq gytghgivvs ilddgieknh pdlagnydpg asfdvndqdp |
| 181 | dpqprytqmn dnrhgtrcag evaavanngv cgvgvaynar iggvrmldge vtdavearsl |
| 241 | glnpnhihiy saswgpeddg ktvdgparla eeaffrgvsq grgglgsifv wasgnggreh |
| 301 | dscncdgytn siytlsissa tqfgnvpwys eacsstlatt yssgnqnekq ivttdlrqkc |
| 361 | teshtgtsas aplaagiial tleanknltw rdmqhlvvqt skpahlnand watngvgrkv |
| 421 | shsygyglld agamvalaqn wttvapqrkc iidiltepkd igkrlevrkt vtaclgepnh |
| 481 | itrlehaqar ltlsynrrgd laihlvspmg trstllaarp hdysadgfnd wafmtthswd |
| 541 | edpsgewvle ientseanny gtltkftlvl ygtapeglpv ppessgcktl tssqacvvce |
| 601 | egfslhqksc vqhcppgfap qvldthyste ndvetirasv capchascat cqgpaltdcl |
| 661 | scpshasldp veqtcsrqsq ssresppqqq pprlppevea gqrlragllp shlpevvagl |
| 721 | scafivlvfv tvflvlqlrs gfsfrgvkvy tmdrglisyk glppeawqee cpsdseedeg |
| 781 | rgertafikd qsal |

What is claimed is:

1. A method of reducing, inhibiting or preventing Aβ and/or AID production by a cell, the method comprising contacting the cell with a BRI2 or BRI3 or a mimic thereof in an amount effective to reduce, inhibit or prevent Aβ and/or AID production by the cell.

2. The method of claim 1, wherein the cell is contacted with a BRI2 or BRI3 that comprises amino acids and/or peptidomimetics equivalent to amino acids 1 to 102 of the human BRI2 protein having the sequence of SEQ ID NO:1 or the human BRI3 protein having the sequence of SEQ ID NO:2,
wherein the BRI2 protein and the BRI3 protein has an amino acid sequence at least 80% homologous to SEQ ID NO:1 and SEQ ID NO:2, respectively.

3. The method of claim 2, wherein the BRI2 or BRI3 is a naturally occurring protein.

4. The method of claim 2, wherein the BRI2 or BRI3 has an amino acid sequence at least 90% homologous to at least a portion of SEQ ID NO:1 or SEQ ID NO:2, respectively.

5. The method of claim 2, wherein the BRI2 or BRI3 has an amino acid sequence at least 95% homologous to at least a portion of SEQ ID NO:1 or SEQ ID NO:2, respectively.

6. The method of claim 2, wherein the BRI2 or BRI3 has an amino acid sequence at least 98% homologous to at least a portion of SEQ ID NO:1 or SEQ ID NO:2, respectively.

7. The method of claim 2, wherein the BRI2 or BRI3 is 100% homologous to at least a portion of SEQ ID NO:1 or SEQ ID NO:2, respectively.

8. The method of claim 2, wherein the BRI2 or BRI3 consists of fewer than 250 amino acids and/or peptidomimetics.

9. The method of claim 2, wherein the BRI2 or BRI3 consists of fewer than 200 amino acids and/or peptidomimetics.

10. The method of claim 2, wherein the BRI2 or BRI3 consists of fewer than 150 amino acids and/or peptidomimetics.

11. The method of claim 2, wherein the BRI2 or BRI3 consists of fewer than 125 amino acids and/or peptidomimetics.

12. The method of claim 2, wherein the BRI2 or BRI3 comprises amino acids and/or peptidomimetics equivalent to amino acids 1 to 102 of the human BRI2 or BRI3 protein having the sequence of SEQ ID NO:1 or SEQ ID NO: 2, respectively.

13. The method of claim 1, wherein the cell is contacted with a BRI2.

14. The method of claim 1, wherein the cell is contacted with a BRI3.

15. The method of claim 1, wherein the cell is contacted with a BRI2 or BRI3 mimic.

16. The method of claim 1, wherein the cell is a neuron.

17. The method of claim 1, wherein the cell is neuronal-like or capable of differentiating into a neuron.

18. The method of claim 1, wherein the cell is in a live mammal.

19. The method of claim 1, wherein the cell is a neuron in a live mammal.

20. The method of claim 1, wherein the cell is in a live human.

21. The method of claim 1, wherein the cell is contacted with a vector comprising a nucleic acid sequence encoding the at least a portion of a BRI2 or BRI3 protein.

22. The method of claim 21, wherein the vector is a viral vector infecting the cell.

23. A method of reducing, inhibiting or preventing Aβ and/or AID production by a cell, the method comprising contacting the cell with a furin in an amount effective to reduce, inhibit or prevent Aβ and/or AID production in the cell.

24. The method of claim 23, wherein the furin comprises an amino acid sequence at least 80% identical to human furin having the sequence of amino acids 108-794 of SEQ ID NO:3.

25. The method of claim 23, wherein the furin comprises an amino acid sequence at least 95% identical to human furin having the sequence of amino acids 108-794 of SEQ ID NO:3.

26. The method of claim 23, wherein the furin is a human furin.

27. The method of claim 23, wherein the cell is contacted with furin protein.

28. The method of claim 27, wherein the furin protein is expressed by a vector comprising a nucleic acid sequence encoding the furin protein.

29. The method of claim 28, wherein the vector is a viral vector.

30. The method of claim 23, wherein the cell is a neuron.

31. The method of claim 23, wherein the cell is neuronal-like or capable of differentiating into a neuron.

32. The method of claim 23, wherein the cell is in a live mammal.

33. The method of claim 23, wherein the cell is a neuron in a live mammal.

34. The method of claim 23, wherein the cell is in a live human.

35. A method of treating a subject having Alzheimer's disease, the method comprising administering to the subject an amount of BRI2 or BRI3 or a mimic thereof effective to treat Alzheimer's disease in the subject.

36. The method of claim 35, wherein the subject is administered a BRI2 or BRI3 that comprises amino acids and/or peptidomimetics equivalent to amino acids 1 to 102 of the human BRI2 protein sequence of SEQ ID NO:1 or the human BRI3 protein sequence of SEQ ID NO:2,
wherein the BRI2 protein and the BRI3 protein has an amino acid sequence at least 80% homologous to SEQ ID NO:1 and SEQ ID NO:2, respectively.

37. The method of claim 36, wherein the BRI2 or BRI3 is a naturally occurring protein.

38. The method of claim 35, wherein the subject is administered a BRI2.

39. The method of claim 35, wherein the subject is administered a BRI3.

40. The method of claim 35, wherein the subject is administered a BRI2 or BRI3 mimic.

41. The method of claim 35, wherein the BRI2 or BRI3 or a mimic thereof is administered directly to the brain of the subject.

42. The method of claim 35, wherein the BRI2 or BRI3 or a mimic thereof is formulated in a pharmaceutical composition that enhances the ability of the BRI2 or BRI3 or mimic thereof to cross the blood-brain barrier of the subject.

43. The method of claim 35, wherein the BRI2 or BRI3 or a mimic thereof is administered in a manner that permits the BRI2 or BRI3 or a mimic thereof to cross the blood-brain barrier of the mammal.

44. A method of treating a subject having Alzheimer's disease, the method comprising administering to the subject an amount of a furin effective to treat Alzheimer's disease in the subject.

45. The method of claim 44, wherein the subject is administered a furin that comprises amino acids and/or peptidomimetics equivalent to a human furin having the sequence of amino acids 108-794 of SEQ ID NO:3,
wherein the furin has an amino acid sequence at least 80% homologous to SEQ ID NO:3.

46. The method of claim 44, wherein the furin is a naturally occurring protein.

47. The method of claim 44, wherein the furin is administered directly to the brain of the subject.

48. The method of claim 44, wherein the furin is formulated in a pharmaceutical composition that enhances the ability of the furin to cross the blood-brain barrier of the subject.

49. The method of claim 44, wherein the furin is administered in a manner that permits the compound to cross the blood-brain barrier of the mammal.

50. A method of determining whether a compound is a mimic of a BRI2 or a BRI3, the method comprising
combining the compound with a functional γ-secretase and a membrane-bound protein comprising a C99, then determining whether the compound inhibits cleavage of the C99 to release Aβ and/or AID,
wherein the compound is a mimic of BRI2 or BRI3 if it inhibits the cleavage of the C99 by the γ-secretase.

51. The method of claim 50, wherein the inhibition of cleavage of the C99 to release Aβ and/or AID is determined by determining whether the compound inhibits release of Aβ and/or AID.

52. The method of claim 50, wherein the inhibition of cleavage of the C99 to release Aβ and/or AID is determined by determining whether the compound causes an increase in the presence of C99.

53. The method of claim 50, wherein the inhibition of cleavage of the C99 to release Aβ and/or AID is determined by determining whether the compound causes a decrease in the presence of C83.

54. The method of claim 50, wherein the inhibition of cleavage of the C99 to release Aβ and/or AID is determined by determining whether the compound causes a decrease in the presence of sAPPα.

55. The method of claim 50, wherein the inhibition of cleavage of the C99 to release Aβ and/or AID is determined by determining whether the compound causes an increase in the presence of sAPPβ.

56. The method of claim 50, wherein the method utilizes an ELISA to quantify a peptide.

57. The method of claim 50, wherein the method utilizes mass spectroscopy.

58. The method of claim 50, wherein the method utilizes a western blot to identify and/or quantify a peptide.

59. The method of claim 50, wherein the compound is designed to mimic a portion of the BRI2 or BRI3 protein comprising amino acids equivalent to amino acids 1 to 102 of the human BRI2 or BRI3 protein having the sequence of SEQ ID NO:1 or SEQ ID NO:2, respectively.

60. The method of claim 59, wherein the compound mimics the three-dimensional structure and/or charge of the portion of the BRI2 or BRI3 protein.

61. The method of claim 59, wherein the BRI2 or BRI3 protein is a BRI2 protein.

62. The method of claim 59, wherein the BRI2 or BRI3 protein is a BRI3 protein.

63. The method of claim 50, wherein the membrane-bound protein comprising a C99 is an amyloid precursor protein (APP).

64. The method of claim 50, wherein the functional γ-secretase and membrane-bound protein comprising a C99 are in a live cell.

65. The method of claim 50, wherein the live cell comprises a genetic construct that activates transcription of a reporter gene upon cleavage of a transgenic APP by γ-secretase.

66. The method of claim 65, wherein the reporter gene is luciferase.

67. The method of claim 65, wherein the transgenic APP further comprises a Gal4 on the cytoplasmic domain of the transgenic APP.

68. The method of claim 64, wherein the live mammalian cell is a neuronal cell.

69. The method of claim 64, wherein the live mammalian cell produces a transgenic APP.

70. The method of claim 69, wherein the live mammalian cell is derived from an HEK293 cell, a HeLa cell, or an N2a cell.

71. A composition comprising a purified BRI2 or BRI3 in a pharmaceutically acceptable excipient.

72. The composition of claim 71, wherein the BRI2 or BRI3 comprises amino acids and/or peptidomimetics equivalent to amino acids 1 to 102 of the human BRI2 protein having the sequence of SEQ ID NO:1 or the human BRI3 protein having the sequence of SEQ ID NO:2,
wherein the BRI2 protein and the BRI3 protein has an amino acid sequence at least 80% homologous to SEQ ID NO:1 and SEQ ID NO:2, respectively.

73. The composition of claim 71, wherein the BRI2 or BRI3 is a BRI2.

74. The composition of claim 71, wherein the BRI2 or BRI3 is a BRI3.

75. The composition of claim 71, wherein the BRI2 or BRI3 consists of fewer than 250 amino acids and/or peptidomimetics.

76. The composition of claim 71, wherein the BRI2 or BRI3 consists of fewer than 200 amino acids and/or peptidomimetics.

77. The composition of claim 71, wherein the BRI2 or BRI3 consists of fewer than 150 amino acids and/or peptidomimetics.

78. The composition of claim 71, wherein the BRI2 or BRI3 consists of fewer than 125 amino acids and/or peptidomimetics.

79. The composition of claim 71, wherein the pharmaceutically acceptable excipient enhances the ability of the BRI2 or BRI3 to cross the blood-brain barrier of the subject.

80. The composition of claim 71, wherein the composition is formulated in unit dosage form for treatment of Alzheimer's disease.

81. A composition comprising a purified furin in a pharmaceutically acceptable excipient.

82. The composition of claim 81, wherein the furin comprises amino acids and/or peptidomimetics equivalent to a human furin having the sequence of amino acids 108-794 of SEQ ID NO:3,
wherein the furin has an amino acid sequence at least 80% homologous to SEQ ID NO:3.

83. The composition of claim 81, wherein the furin is a naturally occurring protein.

84. The composition of claim 81, wherein the pharmaceutically acceptable excipient enhances the ability of the furin to cross the blood-brain barrier of the subject.

85. The composition of claim 81, wherein the composition is formulated in unit dosage form for treatment of Alzheimer's disease.

86. A composition comprising a vector encoding a BRI2 or BRI3 in a pharmaceutically acceptable excipient.

87. The composition of claim 86, wherein the BRI2 or BRI3 comprises amino acids equivalent to amino acids 1 to 102 of the human BRI2 protein having the sequence of SEQ ID NO:1 or the human BRI3 protein having the sequence of SEQ ID NO:2,
wherein the BRI2 protein and the BRI3 protein has an amino acid sequence at least 80% homologous to SEQ ID NO:1 and SEQ ID NO:2, respectively.

88. The composition of claim 86, wherein the pharmaceutically acceptable excipient enhances the ability of the BRI2 or BRI3 to cross the blood-brain barrier of the subject.

89. The composition of claim 86, wherein the composition is formulated in unit dosage form for treatment of Alzheimer's disease.

90. The composition of claim 86, wherein the vector is a virus.

91. A composition comprising a vector encoding a furin in a pharmaceutically acceptable excipient.

92. The composition of claim 91, wherein the furin comprises amino acids equivalent to a human furin having the sequence of amino acids 108-794 of SEQ ID NO:3,
wherein the furin has an amino acid sequence at least 80% homologous to SEQ ID NO:3.

93. The composition of claim 91, wherein the furin is a naturally occurring protein.

94. The composition of claim 91, wherein the pharmaceutically acceptable excipient enhances the ability of the furin to cross the blood-brain barrier of the subject.

95. The composition of claim 91, wherein the composition is formulated in unit dosage form for treatment of Alzheimer's disease.
